(19) United States
(12) Patent Application Publication
(10) Pub. No.: US 2026/0158039 A1
(43) Pub. Date: Jun. 11, 2026

(54) PHARMACEUTICAL COMPOSITION FOR PREVENTING OR TREATING INVOLUNTARY MOVEMENTS RELATED TO DOPAMINERGIC DYSFUNCTION

(71) Applicants: National Cheng Kung University, Tainan (TW); National Chung Cheng University, Chiayi County (TW)

(72) Inventors: Pei-Chun Chen, Tainan (TW); Ching-Yi Chen, Chiayi County (TW)

(21) Appl. No.: 19/405,652

(22) Filed: Dec. 2, 2025

Related U.S. Application Data
(60) Provisional application No. 63/728,695, filed on Dec. 6, 2024.

(51) Int. Cl. A61K 31/549; A61K 9/107; A61K 47/34; A61P 25/14

(52) U.S. Cl. CPC A61K 31/549; A61K 9/1075; A61K 47/34; A61P 25/14

(57) ABSTRACT

The present invention relates to compositions and methods for preventing or treating involuntary movements associated with dopaminergic dysfunction, including L-DOPA-induced dyskinesia. The composition comprises a potassium channel opener and a pharmaceutically acceptable carrier, wherein the carrier is a micelle. In certain embodiments, the potassium channel opener is an ATP-sensitive potassium (K_{ATP}) channel opener such as diazoxide, which may be embedded in or encapsulated by the micelle. The micelle may comprise a block copolymer and exhibit a particle size between 35 nm and 70 nm and/or a zeta potential between −15 mV and −2 mV The composition may be formulated for intranasal administration and provided as a liquid, dry powder, suspension, or combinations thereof. The invention further provides methods for treating involuntary movements by administering the micelle-based potassium channel opener composition to a subject in need, including human subjects, optionally at a dose ranging from 0.08 μg/kg to 120 μg/kg.

### CROSS-REFERENCE TO RELATED APPLICATION

This application claims the benefit of U.S. Provisional Application Ser. No. 63/728,695, filed Dec. 6, 2024. The disclosure of the above application is incorporated herein by reference in its entirety.

### FIELD

The present invention relates to the field of medicine, and specifically to the prevention or treatment of neurodegenerative diseases.

### BACKGROUND

The main pathological hallmark of Parkinson's disease is neurodegeneration in the nigrostriatal pathway, which comprises dopaminergic neurons projecting from the substantia nigra pars compacta (SNpc) to the striatum (STR). When the dopaminergic projections of the nigrostriatal pathway degenerate, motor control is impaired due to dopamine deficiency, thereby leading to the typical symptoms of Parkinson's disease, such as bradykinesia, tremor, and postural instability.

L-DOPA (levodopa) is the first-line therapeutic agent for Parkinson's disease. It can be converted into dopamine by DOPA decarboxylase, thereby stimulating the above-mentioned neurons. Clinically, it is often co-administered with a decarboxylase inhibitor to ensure that more L-DOPA crosses the blood-brain barrier and is converted into dopamine in the brain. Although this treatment can maintain good efficacy for approximately 2 to 5 years, long-term administration of L-DOPA (about 6 to 10 years) may reduce therapeutic effects and induce side effects, including a narrowed therapeutic window, the need for higher doses, and L-DOPA-induced dyskinesia (LID). Such involuntary movements include dystonia, chorea, and athetosis, which can significantly impact the patient's quality of life.

Therefore, there is an urgent need to prevent or treat L-DOPA-induced involuntary movements.

### SUMMARY

To address the aforementioned problems, the present invention provides a composition for preventing or treating an involuntary movement related to dopaminergic dysfunction. The composition comprises: a potassium channel opener and a pharmaceutically acceptable carrier.

In certain embodiments, the involuntary movement related to dopaminergic dysfunction is L-DOPA-induced dyskinesia. In certain embodiments, the composition is formulated for intranasal administration and may be in the form of a liquid, a dry powder, a suspension, or any combination thereof.

In certain embodiments, the potassium channel opener comprises an ATP-sensitive potassium (K_{ATP}) channel opener, such as diazoxide. In certain embodiments, the potassium channel opener is embedded in or encapsulated by the micelle.

In certain embodiments, the pharmaceutically acceptable carrier is a micelle. The micelle may comprise a block copolymer, have a particle size between 35 nm and 70 nm, and/or have a zeta potential of the micelle is between −15 mV and −2 mV. In certain embodiments, the weight ratio of the potassium channel opener to the micelle ranges from 1:100 to 1:1500.

In another aspect, the present invention provides a method for preventing or treating an involuntary movement related to dopaminergic dysfunction, comprising: administering to a subject in need a composition comprising a potassium channel opener and a pharmaceutically acceptable carrier.

In certain embodiments, the involuntary movement related to dopaminergic dysfunction is L-DOPA-induced dyskinesia. In certain embodiments, the composition is administered through the nasal cavity and is provided in the form of a liquid, a dry powder, a suspension, or any combination thereof.

In certain embodiments, the potassium channel opener comprises an ATP-sensitive potassium (K_{ATP}) channel opener, such as diazoxide, and may be embedded in or encapsulated by the micelle.

In certain embodiments, the pharmaceutically acceptable carrier is a micelle comprising a block copolymer, having a particle size between 35 nm and 70 nm, and/or a zeta potential of the micelle is between −15 mV and −2 mV In certain embodiments, the weight ratio of the potassium channel opener to the micelle ranges from 1:100 to 1:1500.

In certain embodiments, the method further comprises administering the composition at a dose from 0.08 μg/kg to 120 μg/kg to a human subject in need.

### BRIEF DESCRIPTION OF THE DRAWINGS

FIG. 1 illustrates the establishment process of the LID mouse model.

FIG. 2 shows the characteristics of the LID mouse model.

FIG. 3A and FIG. 3B illustrate the variation of AIM scores during a L-DOPA therapy, wherein FIG. 3A shows the assessment of forelimb AIM scores, FIG. 3B shows the assessment of axial AIM scores, # represents p<0.05, ## represents p<0.01, differences were evaluated using either Student's t-test or two-way ANOVA and Tukey's post hoc test.

FIG. 4 is a first experimental flowchart.

FIG. 5A is an immunohistochemistry staining image of tyrosine hydroxylase (TH) in striatum (left panel) and substantia nigra (right panel), which is used to observe the differences resulting from different drug treatments.

FIG. 5B is a quantitative graph showing the TH intensity in the striatum under different drug treatments.

FIG. 5C is a quantitative graph showing the number of TH-expressing cells in the substantia nigra under different drug treatments.

FIG. 6A shows the effects of different drug treatments on forelimb AIM scores.

FIG. 6B shows the effects of different drug treatments on axial AIM scores.

FIG. 7A is a flowchart of synthesis and assessment steps for the micelle.

FIG. 7B demonstrates a specific assessment step for the micelle.

FIG. 8A illustrates the synthesis process of the fluorescent molecule (PNA).

FIG. 8B illustrates the synthesis process of the polymer (mPEG-b-PCL).

FIG. 8C illustrates the process of conjugating the fluorescent molecule (PNA) to the polymer (mPEG-b-PCL).

FIG. 9A is a proton NMR spectrum of the polymer (mPEG-b-PCL).

FIG. 9B is a proton NMR spectrum of the micelle (also refer to as PPP or mPEG-b-PCL-PNA).

FIG. 10A to FIG. 10C are calibration curves.

FIG. 11A is an image showing the result of the micelle stability test.

FIGS. 11B and 11C show the quantification results of the micelle stability tests, respectively.

FIG. 12A is a calibration curve of the diazoxide.

FIG. 12B shows a comparison between the permeability coefficients.

FIG. 13 is an image of an olfactory tubercle for illustrating that the diazoxide-loaded micelle can enter the olfactory tubercle via nasal delivery.

FIG. 14 is a second experimental flowchart, wherein five mice (N=5) were used in the control group (CTR), five mice (N=5) in the lesioned group (6-OHDA-saline), ten mice (N=10) in the FAUC-365 treatment group, and ten mice (N=10) in the diazoxide-loaded micelle treatment group (denoted as diazoxide).

FIG. 15A and FIG. 15B are comparative graphs of the axial AIM scores and the forelimb AIM scores respectively for illustrating the therapeutic effect of the diazoxide-loaded micelle. As shown in X-axis of the figures, “CTR” represents the control group, “6-OHDA” represents the lesioned group, “6-OHDA/Dia+” represents the diazoxide-loaded micelle treatment group, “6-OHDA/FAUC” represents the FAUC-365 treatment group, * represents p<0.05, ** represents p<0.01, differences were assessed by one-way ANOVA and Tukey's post hoc test.

FIG. 16A is an immunohistochemistry staining image of mice striatum and substantia nigra.

FIG. 16B is a quantitative graph of TH fluorescence intensity derived from the immunohistochemistry staining image, wherein “L” corresponds to the lesioned side as shown in FIG. 16A, and “NL” corresponds to the non-lesioned side in FIG. 16A.

FIG. 16C is a quantitative graph of substantia nigra neurons, wherein “L” corresponds to the lesioned side as shown in FIG. 16A, and “NL” corresponds to the non-lesioned side in FIG. 16A.

FIG. 17, comprising a fluorescent image (left) and a reference diagram (right), illustrates the exact location of the micelle in the mouse brain after being delivered through the nasal cavity.

FIG. 18A and FIG. 18B show the tolerance test results of insulin and glucose, respectively.

### DETAILED DESCRIPTION

The present invention provides a composition for preventing or treating an involuntary movement related to dopaminergic dysfunction, comprising: a potassium channel opener (K⁺ channel opener) and a pharmaceutically acceptable carrier. As used herein, “treating” includes alleviating symptoms, preventing deterioration, slowing disease progression or promoting recovery.

In some embodiments, the potassium channel opener is a compound that, directly or indirectly, induces a conformational change in an ion channel upon contact, and thereby promotes the passage of potassium ions across the cell membrane on which the ion channel is expressed. The ion channel may be expressed on any mammalian cells, including but not limited to the neural cells (such as neurons), cardiomyocytes, skeletal muscle cells, renal epithelial cells, pancreatic 0-cells, or vascular endothelial cells. In certain embodiments, the ion channel comprises a cation channel; the cation channel maybe a potassium channel, and the potassium channel may be an ATP-sensitive potassium (K_{ATP}) channel.

Accordingly, in some embodiments, the potassium channel opener is an ATP-sensitive potassium channel opener. In some embodiments, the ATP-sensitive potassium channel opener comprises Diazoxide, Pinacidil, Nicorandil, Minoxidil, Retigabine, or any combination thereof.

In some embodiments, the pharmaceutically acceptable carrier is a particulate carrier, and the surface of the particulate carrier may be charged or neutral.

In some embodiments, the particulate carrier comprises a polymer chain that can be a block copolymer, which may include a hydrophilic block and/or a hydrophobic block. It is to be understood that, in some embodiments, the polymer chain comprises a hydrophilic segment (tail) and a hydrophobic segment (tail). In some embodiments, the particulate carrier has a core-shell structure, wherein the core-shell structure includes an inner core portion and an outer shell portion, the inner core portion being formed from the hydrophobic segment, the outer shell portion being formed from the hydrophilic segment.

In some embodiments, the hydrophilic block comprises polyethylene glycol (PEG), polyvinylpyrrolidone (PVP), or any combination thereof, without limitation.

In some embodiments, the hydrophobic block comprises polylactic acid (PLA), poly-F-caprolactone (PCL), polyphenylene oxide (PPO), or any combination thereof, without limitation.

In some embodiments, the composition of the particulate carrier further comprises an environmentally responsive polymer, which may include a temperature-responsive polymer, a pH responsive polymer, a light responsive polymer, an ion responsive polymer, or any combination thereof, without limitation. In some embodiments, the temperature-responsive polymer is poly (N-isopropylacrylamide), and the pH responsive polymer is poly(2-(diisopropylamino)methyl methacrylate) (PDPA). It is to be understood that the environmentally responsive polymer can constitute one of the blocks of the block copolymer.

In some embodiments, the composition of the particulate carrier further comprises a fluorescent compound containing an imide structure, such as 1,8-naphthalimide or a derivative thereof. It is to be understood that, the fluorescent compound can be attached to the tail of the polymer chain or can constitute a block of the block copolymer, without limitation.

In some embodiments, the block copolymer is mPEG-b-PCL, or mPEG-b-PCL-PNA.

In some embodiments, the potassium channel opener is distributed within the interior or on the surface of the particulate carrier. In some embodiments, the particle size of the particulate carrier ranges from 5 nm to 500 nm.

In some embodiments, the particulate carrier comprises a micelle, a liposome, polymeric nanoparticles, a nanogel, a microgel, a polymersome, or any combination thereof.

In some embodiments, the potassium channel opener is embedded in or encapsulated by the micelle. In some embodiments, the particle size of the micelle ranges from 35 to 70 nm, for example 35, 40, 45, 50, 55, 60, 65, or 70 nm. In some embodiments, the zeta potential of the micelle ranges from −50 to −1 mV, or preferably from −15 to −2 mV, for example −15, −14, −13, −12, −11, −10, −9, −8, −7, −6, −5, −4, −3, or −2 mV.

In some embodiments, the weight ratio of the potassium channel opener to the micelle ranges from 1:(100 to 1500), or ranges from 1:(200 to 1000). For example, the weight ratio of the potassium channel opener to the micelle can be 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1050, 1:1100, 1:1150, 1:1200, 1:1250, 1:1300, 1:1350, 1:1400, 1:1450, or 1:1500.

In some embodiments, the composition is administered to a subject in an effective dose, and the subject is a mammal, such as human (Homo sapiens), dog (Canis lupus familiaris), cat (Felis catus), cattle (Bos taurus), pig (Sus scrofa domesticus), horse (Equus ferus caballus), mouse (Mus musculus), rat (Rattus norvegicus), rabbit (Oryctolagus cuniculus), or monkey (Macaca mulatta), without limitation.

In some embodiments, the subject is a mouse, and the effective dose is selected from a range of 0.001 mg/kg to 1 mg/kg. In some preferred embodiments, the subject is a mouse, and the effective dose is selected from a range of 0.01 mg/kg to 0.1 mg/kg, for example, the effective dose can be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, or 0.1 mg/kg.

In some embodiments, based on the human equivalent dose (HED) conversion, the subject is a human, and the effective dose is selected from a range of 0.08 μg/kg to 120 pg/kg. In some preferred embodiments, the effective dose is selected from a range of 0.8 μg/kg to 12 μg/kg, for example, the effective dose can be 0.81 μg/kg, 1.62 μg/kg, 2.43 μg/kg, 3.24 μg/kg, 4.06 μg/kg, 4.87 μg/kg, 5.68 μg/kg, 6.49 μg/kg, 7.30 μg/kg, 8.11 μg/kg, or 10.00 μg/kg.

The aforementioned dopamine is a neurotransmitter that plays a role in stimulating, inhibiting, or providing feedback within a nervous system of a human or animal, thereby inducing physiological or psychological changes. In a physiological environment, the presynaptic terminal of a first neuron can release the dopamine, which diffuses across the synaptic cleft and is received by the postsynaptic terminal of a second neuron. In some embodiments, the dopamine includes L-3,4-dihydroxyphenylalanine (L-DOPA, levodopa) or D-3,4-dihydroxyphenylalanine (D-DOPA). In some preferred embodiments, the dopamine is L-DOPA.

The term “dopaminergic dysfunction” as used herein refers to an interruption of dopamine secretion and reception between neurons, or a change in dopamine concentration in the synaptic cleft, resulting in physiological or psychological changes or abnormalities in the subject. The physiological or psychological abnormalities comprise, for example, motion abnormalities, cognitive abnormalities, and emotional abnormalities. The dopaminergic dysfunction may be primary or secondary.

The aforementioned motion abnormalities include “involuntary movements/abnormal involuntary movements (AIMs)”. The involuntary movements comprise dyskinesia, and the dyskinesia can be selected from one or more of the following: tremor, chorea, akathisia, tics, myoclonus, and dystonia.

It should be further understood that reception of dopamine by a neuron is mediated by a dopamine receptor. The dopamine receptor includes D1-like receptor family, comprising dopamine D1 receptor and dopamine D5 receptor, and D2-like receptor family, comprising dopamine D2 receptor, dopamine D3 receptor, and dopamine D4 receptor.

In some embodiments, the dopaminergic dysfunction is caused by drug therapy. For example, the involuntary movement related to dopaminergic dysfunction may arise from treatment for Parkinson's disease. specifically, the involuntary movement related to the dopaminergic dysfunction includes L-DOPA-induced dyskinesia (LID), which is caused by long-term treatment with L-DOPA in the subject.

Furthermore, the L-DOPA-induced dyskinesia results from abnormal activation of medium spiny neurons (MSNs) located in the striatum (STR), wherein expression of D1-like and D2-like receptor family in the striatum of a subject with Parkinson's disease is relatively increased compared with that in a healthy subject.

In some embodiments, the composition reduces the expression of the dopamine receptor in neurons, thereby preventing or treating the L-DOPA-induced dyskinesia. In some preferred embodiments, because the dopamine D3 receptor belongs to the D2-like receptor family, has high structural similarity within the family, and is primarily distributed in the limbic system, modulation of the dopamine D3 receptor can be used to treat the L-DOPA-induced dyskinesia.

In some embodiments, the composition is formulated for administration through a nasal cavity.

In some embodiments, the composition may be provided in the form of a liquid, a dry powder, a suspension, or any combination thereof, including, without limitation, a cream, such that the composition is suitable for nasal administration. In some embodiments, the composition may be a nasal inhaler or a nasal spray.

In some embodiments, the composition can be used in the preparation of a drug for preventing or treating the involuntary movement related to the dopaminergic dysfunction, wherein the route of administration comprises administration via the nasal cavity.

Embodiments are provided below for further illustrating the present invention.

### Experiment 1: Potassium Channel Opener can be Used for Treating L-DOPA-Induced Dyskinesia

In this experiment, abnormal involuntary movements (AIMs) are assessed, including two types of AIMs: axial dyskinesia and limb dyskinesia. Axial dyskinesia comprises contralateral twisting and dystonic posturing, and limb dyskinesia comprises forelimb shaking and jerky movements. For axial AIMs, a first basal severity score is multiplied by an AIM amplitude score. The first basal severity score can be represented as: 0: no turning; 1: turning less than 30 seconds; or 2: turning more than 30 seconds. The AIM amplitude scale can be represented as: 0: no turning; 1: turning fewer than 10 times; 2: turning 11-20 times; 3: turning 21-30 times; and 4: turning 31-40 times. For limb AIMs, a second basal severity score is multiplied by a postural score. The second basal severity score can be represented as: 0: no shaking; 1: shaking less than 30 seconds; 2: shaking more than 30 seconds. The postural score can be represented as: 0: no flapping; 1: locomotion flapping and paw shaking; 2: not only the paw but the whole arm shaking; and 3: arm shaking accompanied by shoulder-back movement. In addition, contralateral rotations (number of full turns) can be evaluated to determine whether D3 receptor (D3R) manipulation affects L-DOPA-evoked motor activity.

In this experiment, an animal model is first established using 8-week-old male C57BL/6 mice. Referring to FIG. 1, a PD mouse (Parkinson's disease mouse) model is established by unilateral injection of 6-OHDA (6-hydroxydopamine) using a 10-μL syringe, according to the following stereotaxic coordinates relative to bregma: anteroposterior +0.14 mm, lateral +0.21 mm, and dorsoventral −3.5 mm, with injections carried out over a period of 3 weeks. Subsequently, an LID mouse (L-DOPA-induced dyskinesia mouse) model is established by intraperitoneal injection of L-DOPA into the PD mouse once daily for 2 weeks.

As shown in FIG. 1, at week 0, mice in the control group receive injections of saline containing 0.02% ascorbic acid into the striatum of both the left and right cerebral hemispheres. In the experimental group, to induce a lesion in the left cerebral hemisphere (lesioned side), mice are injected with 4 μg/μL of 6-OHDA in saline containing 0.02% ascorbic acid into the left striatum. To avoid inducing a lesion in the right cerebral hemisphere (non-lesioned side), mice are injected with saline containing 0.02% ascorbic acid into the right striatum. Subsequently, from week 3 to week 5, both the control group and experimental group receive intraperitoneal injections of L-DOPA and benserazide (a decarboxylase inhibitor). Beginning at week 3, AIM tests are performed on day 1, day 6, and day 13, respectively.

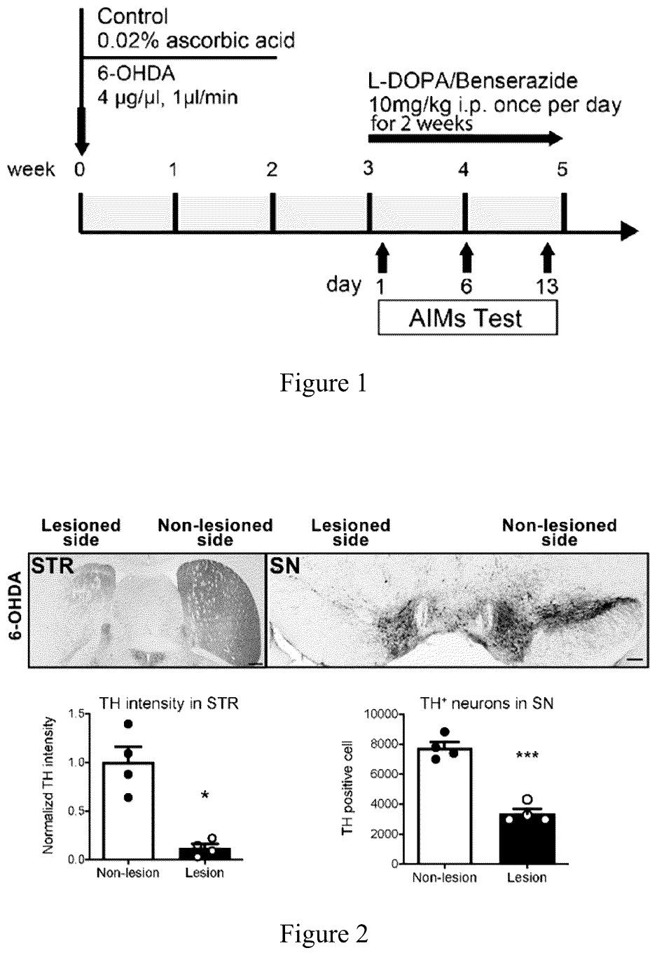

As shown in FIG. 2, which presents immunohistochemistry staining images of the striatum (STR) and substantia nigra (SN), the figure illustrates the content and distribution of tyrosine hydroxylase (TH) in TH-positive neurons. A more intense staining indicates a higher number of TH-positive neurons.

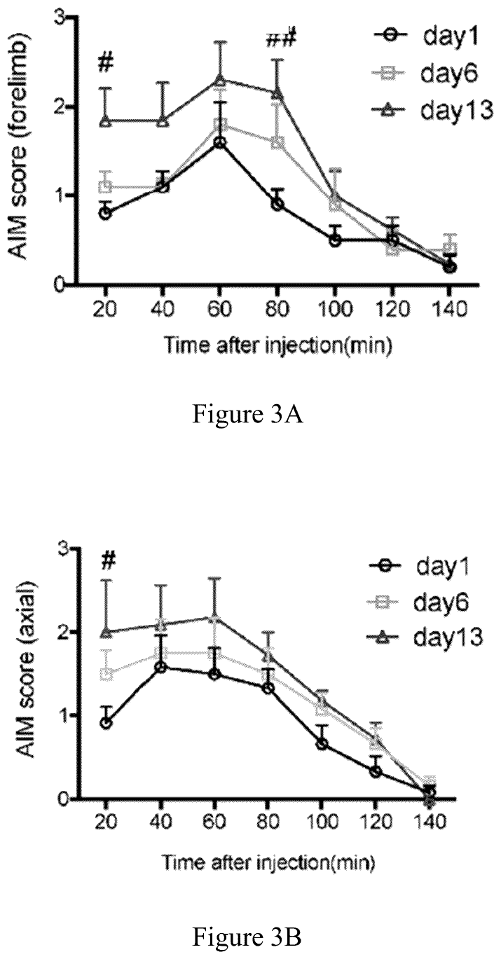

Referring to FIG. 2, the staining intensity in the 6-OHDA-induced lesioned side is reduced, indicating a decreased number of TH-positive neurons. This result reflects reduced dopamine secretion, a hallmark of Parkinson's disease. As shown in FIGS. 3A and 3B, over time, AIM tests show that both forelimb AIM scores and axial AIM scores gradually increase under L-DOPA treatment, and the involuntary movements worsen.

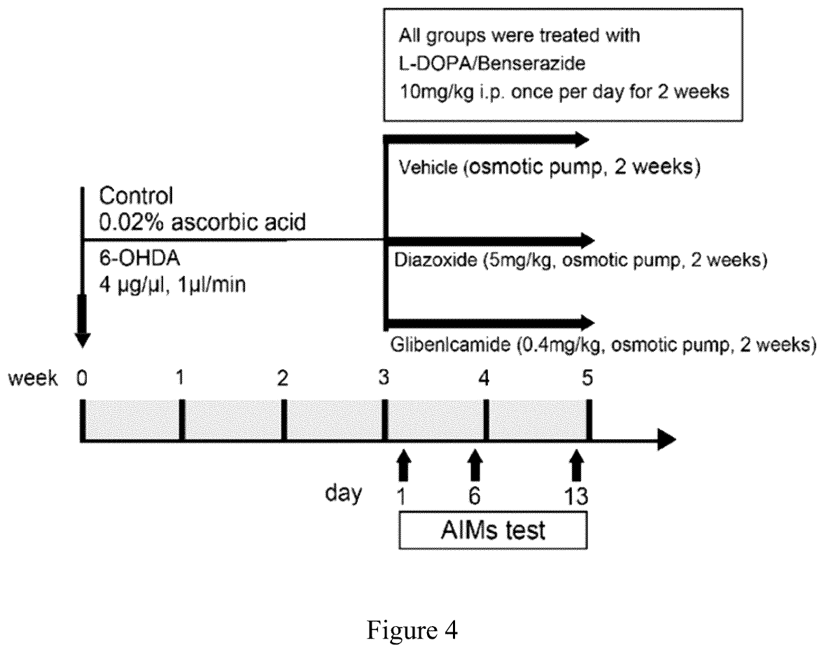

With reference to FIG. 4, the figure demonstrates the effect of potassium channels in LID mice. In different experimental groups, mice receive: group (1), vehicle (88% PEG, 10% DMSO, and 2% Tween 80); group (2), potassium channel opener diazoxide (Diz); and group (3), potassium channel inhibitor glibenclamide (Gbc). Vehicle or drug is administered starting at week 3 for 2 weeks. Using the LID mouse model, vehicle or drug is administered from week 3 to week 5 using implanted mini-osmotic pumps (Alzet® 1002). The catheter is positioned according to the coordinates anteroposterior +0.14 mm, lateral +0.21 mm, and dorsoventral −3.5 mm, corresponding to the striatum. The infusion rate is 0.26 μL/h.

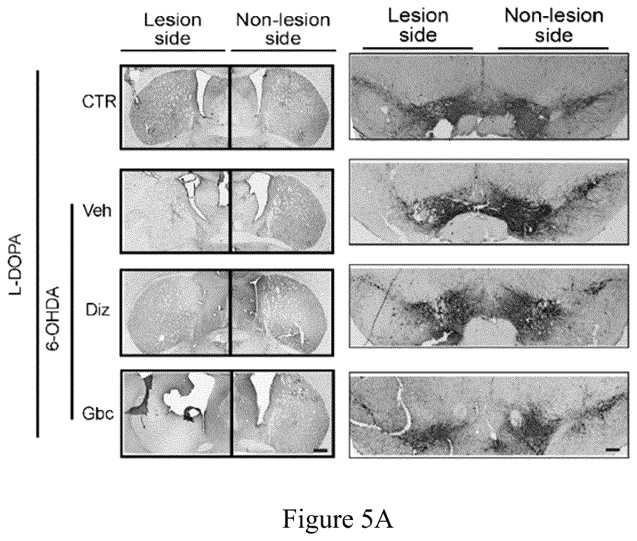

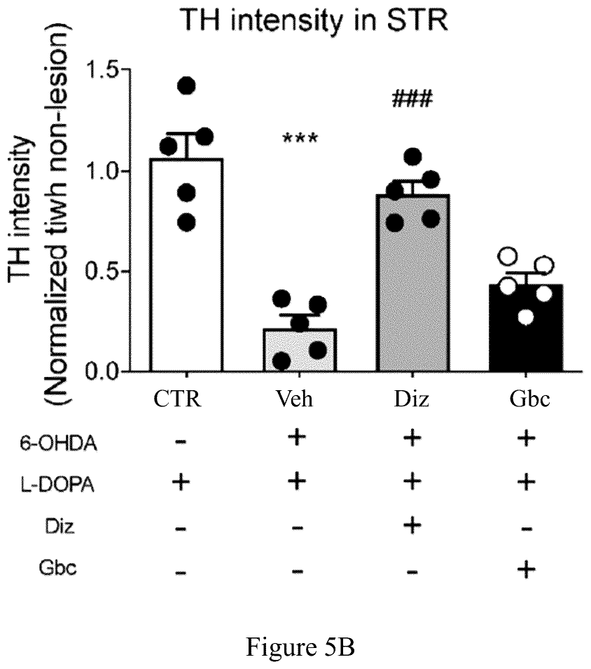

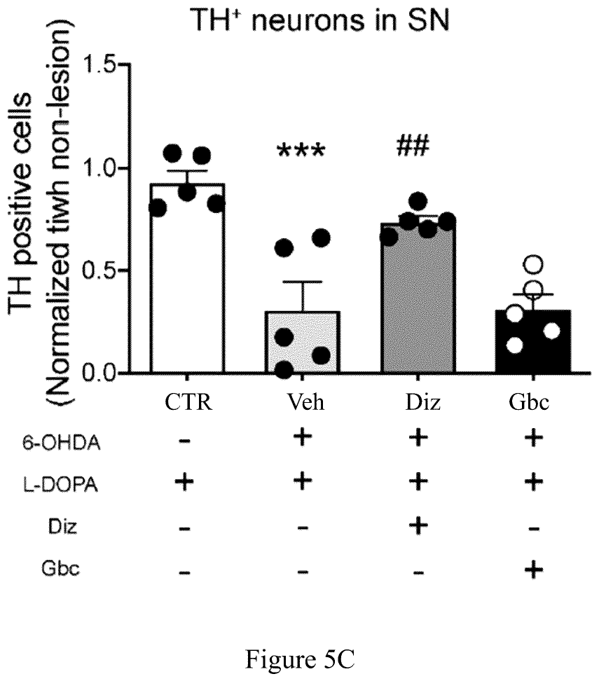

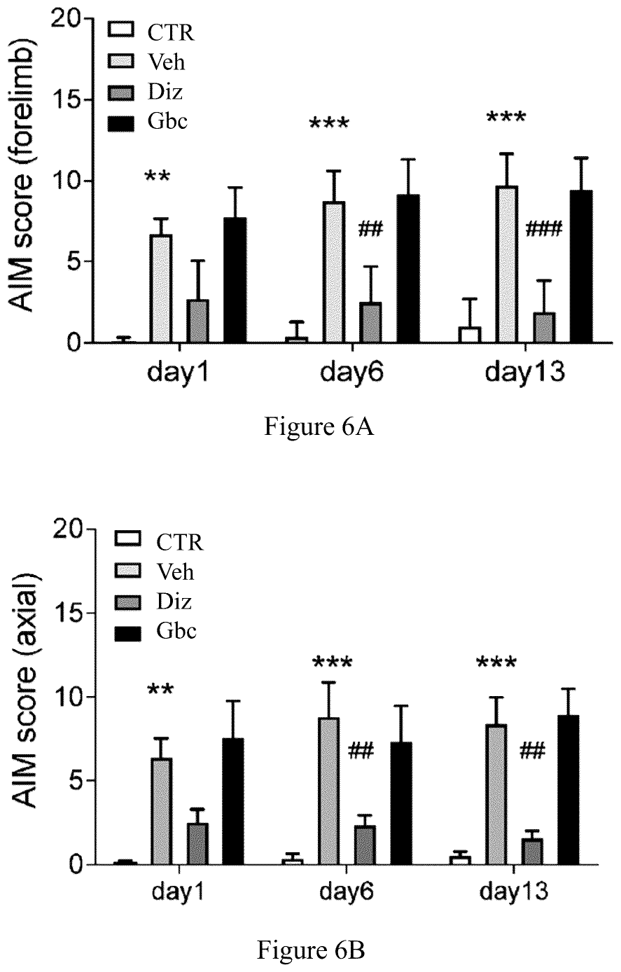

Referring to FIG. 5A, which presents immunohistochemistry staining of TH-positive neurons, and to FIGS. 5B and 5C, it is apparent that diazoxide administration promotes recovery of TH-positive neurons to levels close to those of the control group (CTR). Furthermore, referring to FIGS. 6A and 6B, AIM tests show that AIM scores in group (2) are significantly lower than those in groups (1) and (3), and are close to levels observed in healthy controls.

In summary, direct administration of a potassium channel opener into the striatum reduces AIM scores. In other words, L-DOPA-induced involuntary movements are reduced by administration of the potassium channel opener.

### Experiment 2: Preparation of a Micelle, Encapsulation of the Potassium Channel Opener in the Micelle, and the Interaction of the Potassium Channel Opener-Loaded Micelle with the Nasal Mucosal Cells

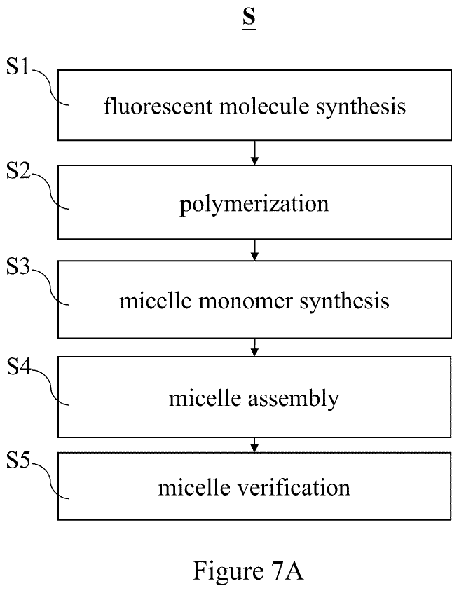

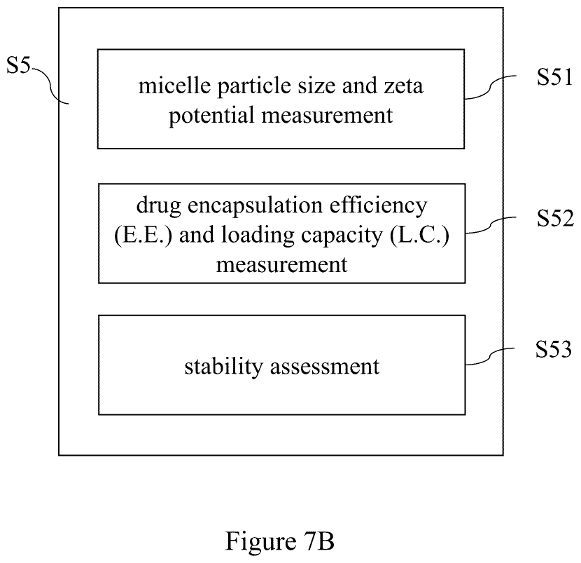

Referring to FIG. 7A, the micelle synthesis and verification process includes steps S1-S5, comprising: a fluorescent molecule synthesis step S1, a polymerization step S2, a micelle monomer synthesis step S3, a micelle assembly step S4, and a micelle verification step S5.

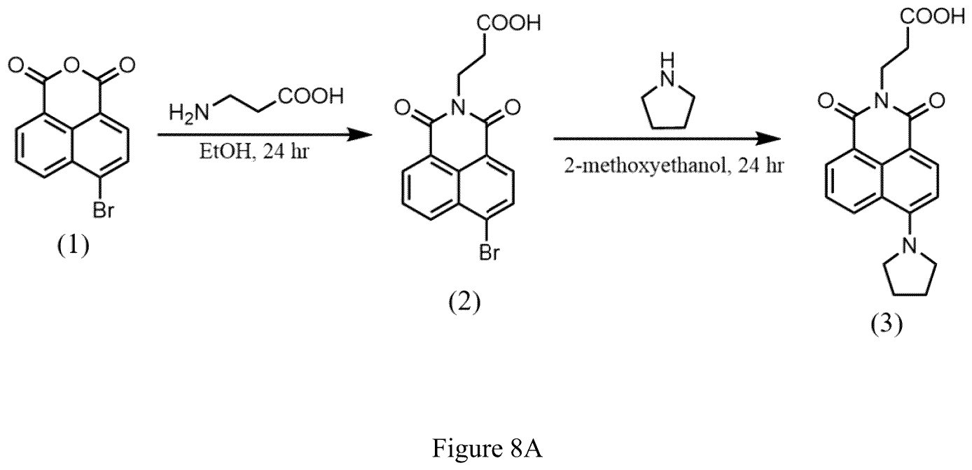

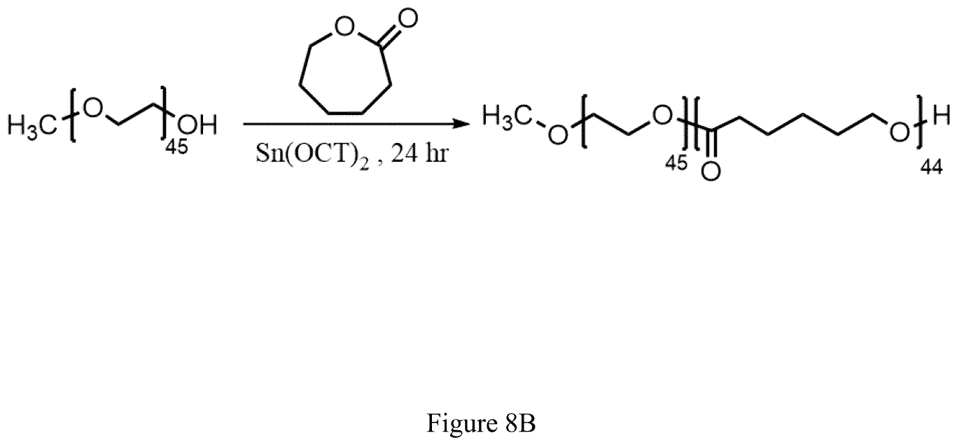

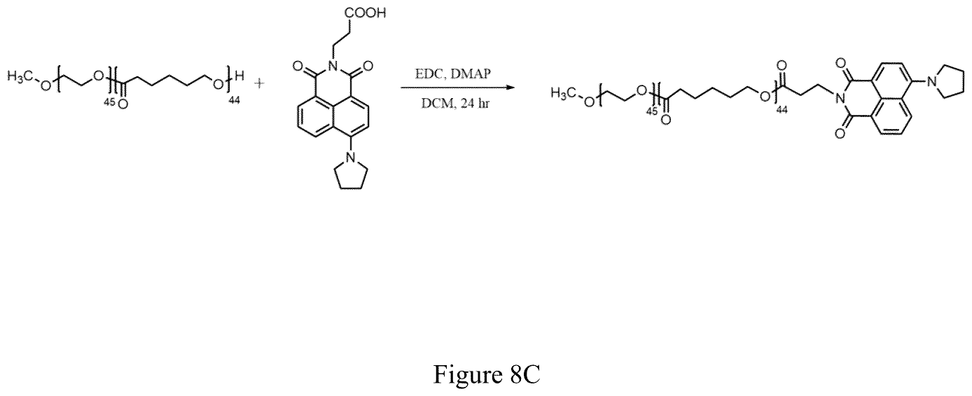

In fluorescent molecule synthesis step S1, the reaction shown in FIG. 8A is carried out to synthesize the fluorescent molecule monomer 3-(1,3-dioxo-6-(pyrrolidin-1-yl)-1H-benzo[de]isoquinolin-2(3H)-yl) propanoic acid (PNA).

Specifically, 2 g (7.2 mmol) of 4-bromo-1,8-naphthalic anhydride and 0.965 g (10.8 mmol) of P-alanine are dissolved in 40 mL of anhydrous ethanol and placed in a three-neck flask equipped with a reflux condenser. The mixture is subjected to an oil bath under a nitrogen atmosphere at 72° C. and 700 rpm for 24 hours to obtain a crude product. The crude product is collected by vacuum filtration to obtain a white product, which is repeatedly washed with anhydrous ethanol and then placed in a vacuum oven at 60° C. for 24 hours to obtain a white solid. Completion of the reaction can be confirmed by thin-layer chromatography (TLC) (DCM/MeOH=8/1, v/v), and the structure and purity of the white solid can be confirmed by ¹H NMR (400 MHz, DMSO-d₆).

Subsequently, 1 g (2.87 mmol) of the white solid and 0.245 g (3.45 mmol) of pyrrolidine are dissolved in 25 mL of 2-methoxyethanol and placed in a three-neck flask equipped with a reflux condenser. The mixture is heated and refluxed at 120° C. for 48 hours. After reaction completion is confirmed by TLC (DCM/MeOH=8/1, v/v), the reaction mixture is cooled to room temperature, and 150 mL of sodium chloride solution is added. The mixture is extracted with 200 mL of ethyl acetate three times, the combined organic layers are dried over anhydrous sodium sulfate, and then filtered. The filtrate is concentrated by rotary evaporation to remove ethyl acetate, and 40 mL of anhydrous ethanol is added and heated to boiling, followed by recrystallization in an ice bath. The resulting crystals are placed in a vacuum oven at 50° C. for 24 hours to obtain an orange powder. The structure and purity of the orange powder can be confirmed by ¹H NMR (400 MHz, DMSO-d₆).

In polymerization step S2, the reaction shown in FIG. 8B is carried out to synthesize the polymer mPEG-b-PCL.

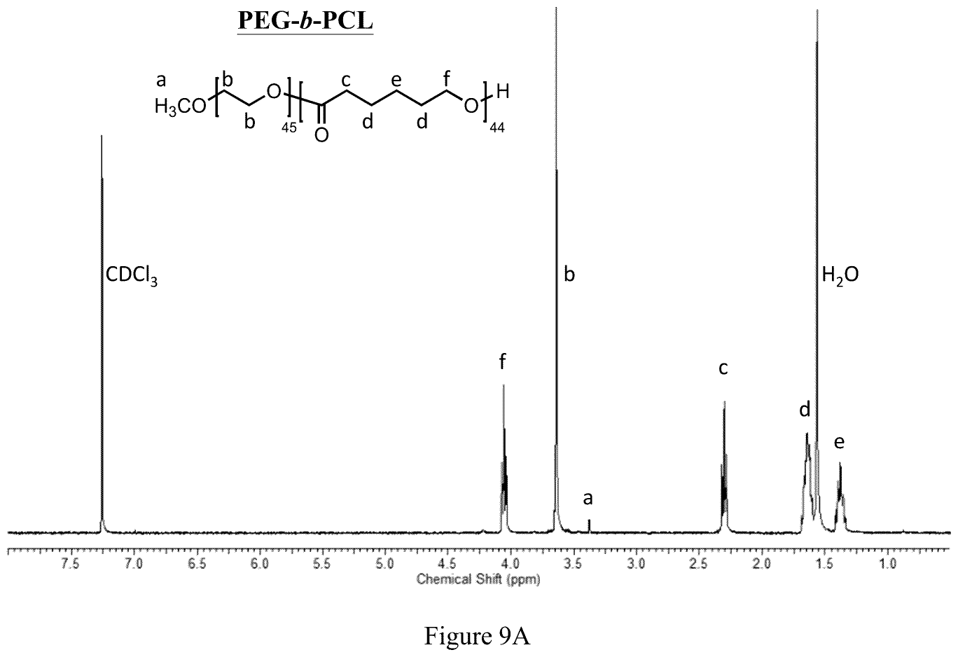

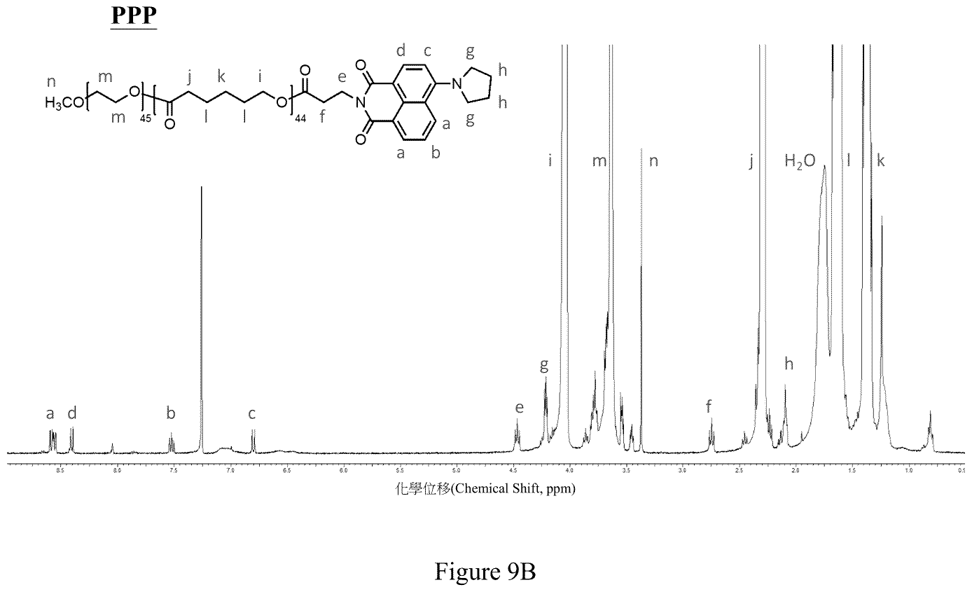

Three grams of mPEG are placed in a Schlenk flask and dried in a vacuum oven at 60° C. for 24 hours. After cooling, 7.54 g (66 mmol) of F-caprolactone and 0.105 g (0.259 mmol) of Sn(Oct)₂ (stannous 2-ethylhexanoate) are added under a nitrogen atmosphere. The mixture is reacted in an oil bath at 124° C. for 24 hours to obtain a yellow viscous product. After cooling in an ice bath, a small amount of tetrahydrofuran (THF) is added, and the mixture is stirred in diethyl ether to precipitate a white solid. The white solid is collected by vacuum filtration, redissolved in a small amount of TIF, and reprecipitated with cold diethyl ether two to three times. The reprecipitated white solid is then placed in a vacuum oven at room temperature for 24 hours to obtain a white granular product. The structure and purity of the white granular product can be confirmed by ¹H NMR (400 MHz, CDCl₃), as shown in FIG. 9A.

In micelle monomer synthesis step S3, a Steglich esterification reaction, as depicted in FIG. 8C, is carried out to synthesize the micelle monomer poly (ethylene glycol)-b-poly(ε-caprolactone)-3-(1,3-dioxo-6-(pyrrolidin-1-yl)-1H-benzo[de]isoquinolin-2(3H)-yl) propanoic acid, hereinafter referred to as PPP or mPEG-b-PCL-PNA.

Three grams (0.426 mmol) of dry mPEG-b-PCL are placed in a Schlenk flask, and 0.026 g (0.213 mmol) of 4-dimethylaminopyridine (4-DMAP) is added. The Schlenk flask is evacuated and purged with nitrogen, and 8 mL of dichloromethane (DCM) is added to dissolve the polymer under a nitrogen atmosphere. Separately, 0.26 g (0.768 mmol) of the fluorescent molecule PNA and 0.204 g (1.064 mmol) of the coupling reagent EDC (1-ethyl-3-(3-dimethylaminopropyl) carbodiimide) are dissolved in a vial under nitrogen. The solution from the vial is then added into the Schlenk flask via a syringe, and the reaction is carried out at 30° C. for 24 hours to obtain a crude product.

The crude product is dialyzed against deionized (DI) water for 48 hours, followed by dialysis against THF for an additional 48 hours. The dialysate is then dropped into ice-cold diethyl ether with vigorous stirring to precipitate a yellow solid. The yellow solid is collected by vacuum filtration, redissolved in a small amount of TIF, and reprecipitated with cold diethyl ether two to three times. The reprecipitated yellow solid is placed in a vacuum oven at room temperature for 24 hours to obtain a yellow granular product. The structure and purity of the yellow granular product can be confirmed by 1H NMR (400 MHz, CDCl₃), as shown in FIG. 9B.

In micelle assembly step S4, the micelle monomers described above undergo self-assembly to form micelles or drug-loaded micelles.

To prepare micelles, 3 mg of PPP are dissolved in 0.6 mL of THF to obtain a mixed solution, which is added via syringe into phosphate-buffered saline (PBS) under continuous rapid stirring at a rate of 25 μL/min, allowing the micelle monomers to self-assemble and form a first micelle solution at a concentration of 1 mg/mL. The first micelle solution is dialyzed against 0.001 M PBS using a dialysis membrane (MWCO 3500) to remove THF, and then filtered through a 0.45 μm PTFE membrane to obtain a second micelle solution.

To prepare drug-loaded micelles, diazoxide is dissolved in dimethyl sulfoxide (DMSO) to prepare a drug solution at a concentration of 5 mg/mL. To investigate different micelle-to-drug weight ratios (10:1, 20:1, and 30:1), 3 mg of PPP are dissolved in corresponding volumes of THF to obtain mixed solutions of 0.54 mL, 0.57 mL, and 0.58 mL, respectively. Then, 0.06 mL of the drug solution is mixed with 0.54 mL of the mixed solution (feed weight ratio 10:1), 0.03 mL of the drug solution with 0.57 mL of the mixed solution (feed weight ratio 20:1), or 0.02 mL of the drug solution with 0.58 mL of the mixed solution (feed weight ratio 30:1). Subsequently, 2.4 mL of 0.01 M PBS is added, and the mixture is ultrasonically homogenized at 750 W×25% (9 s on/4 s off) for 15 minutes at 4° C. to prepare drug-loaded micelle solutions with the corresponding micelle/drug weight ratios. The drug-loaded micelle solutions are then dialyzed against 0.001 M PBS using a dialysis membrane (MWCO 3500) to remove THF, DMSO, and free diazoxide, yielding drug-loaded micelle solutions containing diazoxide-loaded micelles.

Referring to FIG. 7B, micelle verification step S5 includes micelle particle size and zeta potential measurement step S51. The particle size and zeta potential of micelles in the second micelle solution are measured using a dynamic light scattering (DLS) particle size analyzer. The measurement parameters are set as follows: scattering angle 90°, temperature 25° C., and temperature equilibration time 2 minutes. During measurement, 1 mL of the second micelle solution is placed in a cuvette, and the average of three measurements is taken as the measured value. Referring to Table 1, the properties of the micelles and diazoxide-loaded micelles are summarized; in Table 1, the diazoxide-loaded micelles are designated as “PPP-D.”

TABLE 1

| | Feed weight | Particle | Polydispersity | Zeta |
|---|---|---|---|---|
| | ratio | size | index | potential |
| | (PPP:D) | (nm) | (PDI) | (mV) |
| Micelle | — | 44.2 ± 0.08 | 0.145 ± 0.024 | −9.3 ± 0.07 |
| PPP-D | 10:1 | 56.9 ± 0.11 | 0.205 ± 0.019 | −5.7 ± 0.16 |
| PPP-D | 20:1 | 53.3 ± 0.03 | 0.139 ± 0.016 | −6.2 ± 0.12 |
| PPP-D | 30:1 | 51.5 ± 0.03 | 0.127 ± 0.027 | −12.1 ± 0.0 |

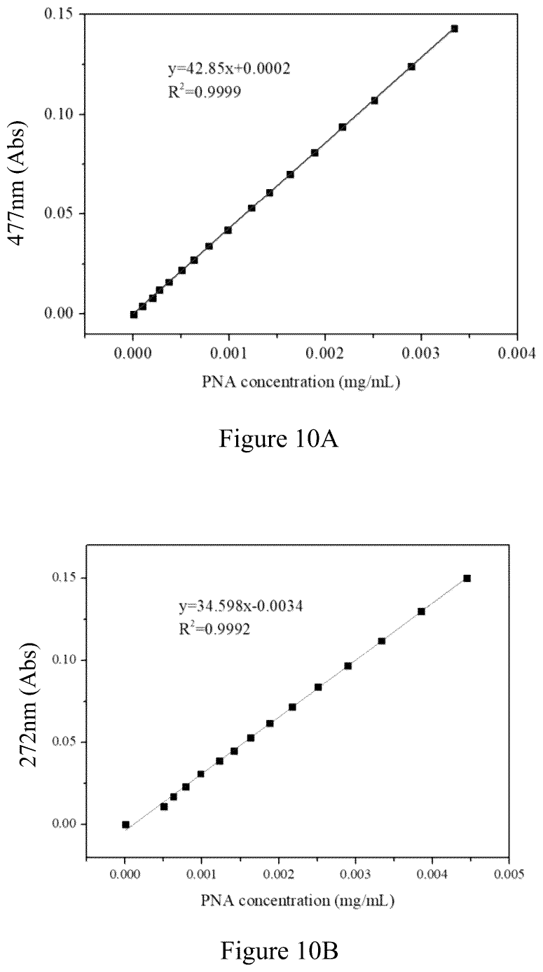

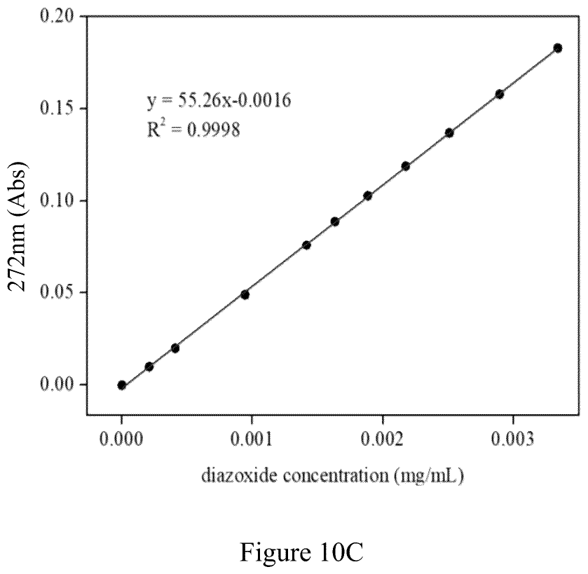

Referring to FIG. 7B, the micelle verification step S5 includes the drug encapsulation efficiency (E.E.) and loading capacity (L.C.) measurement step S52. A quantified amount of diazoxide-loaded micelles are subjected to lyophilization and weighed respectively, wherein both the lyophilized samples are then dissolved in a known volume of dimethyl sulfoxide (DMSO), and their absorbance is measured using a UV-visible spectrophotometer (Shimadzu UV-2600) at a specific wavelength, such as 272 nm corresponding to diazoxide. Using calibration curves of the fluorescent molecule PNA in DMSO (at 447 nm and 272 nm, as shown in FIGS. 10A and 10B, respectively) and of diazoxide in DMSO (as shown in FIG. 10C), the respective concentrations are calculated. The related data are further determined according to Equation 1 and Equation 2. Referring to Table 2, the properties of the diazoxide-loaded micelles (PPP-D) are shown.

$$E . E . ( \% ) = \frac{drug loading amount}{total drug amount} \times 100 \% \qquad Equation 1$$

$$L . C . ( \% ) = \frac{drug loading amount}{total weight of micelle molecular} \times 100 \% \qquad Equation 2$$

TABLE 2

| | Feed weight ratio | | |
|---|---|---|---|
| No. | (PPP:D) | E.E (%) | L.C. (%) |
| 1 | 10:1 | 18.36 ± 2.06 | 1.02 ± 0.09 |
| 2 | 20:1 | 11.14 ± 0.51 | 0.47 ± 0.11 |
| 3 | 30:1 | 7.95 ± 0.20 | 0.36 ± 0.13 |

As shown in Table 2, the diazoxide-loaded micelles labeled as No. 1 exhibited the highest drug loading capacity (L.C.). Therefore, unless otherwise specified, the subsequent experiments are primarily conducted using diazoxide-loaded micelles prepared at the feed weight ratio (PPP:D) of 10:1.

Referring to FIG. 7B, the micelle evaluation step S5 includes a stability assessment step S53. On one hand, a quantified amount of diazoxide-loaded micelles are placed at 4° C. and 37° C. for up to 28 days, during which changes in appearance, color, and turbidity are recorded. On the other hand, dynamic light scattering (DLS) is used to monitor changes in particle size and zeta potential of micelles on days 1, 7, 14, 21, and 28.

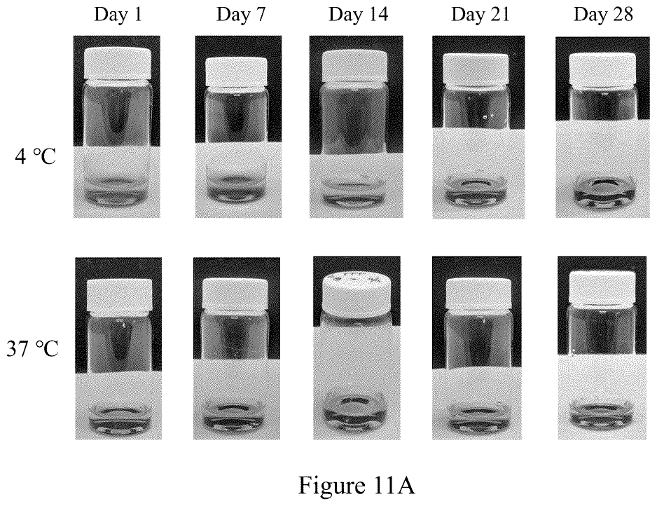

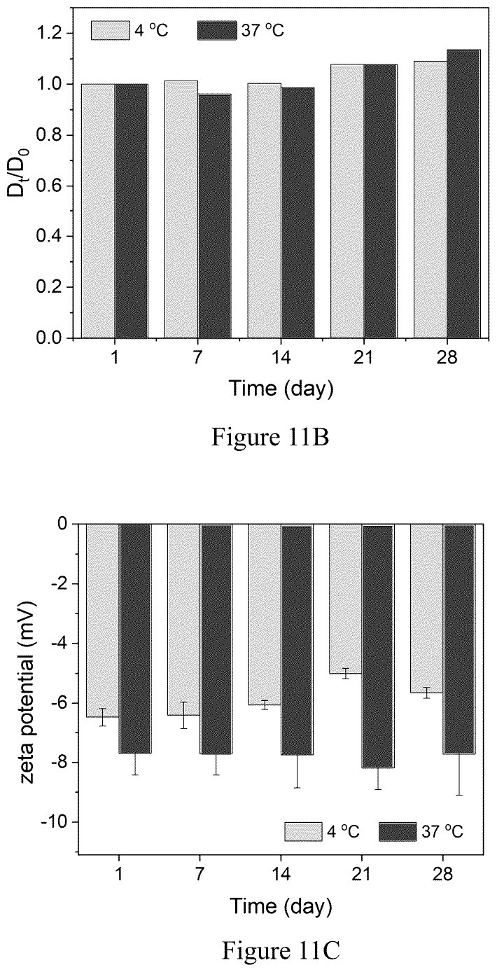

Regarding changes in appearance, color, and turbidity, as shown in FIG. 11A, it can be clearly observed that during the 28-day period, the solutions of diazoxide-loaded micelles remained clear at both 4° C. and 37° C. For particle size and zeta potential of the micelles, refer to FIG. 11B; the ratio of Dₜ to D₀ remained approximately 1.0 over 28 days at both temperatures, indicating good dispersibility and stability. Meanwhile, as shown in FIG. 11C, the zeta potential of diazoxide-loaded micelles remained relatively constant at both 4° C. and 37° C. Therefore, the micelles, prepared using the aforementioned method, exhibit considerable stability.

Subsequently, in this experiment, a micelle permeation test is conducted to confirm that diazoxide-loaded micelles are more suitable for penetrating a nasal epithelial cell layer model compared to free diazoxide.

For the establishment of a nasal epithelial cell layer model, the human nasal septum squamous cell carcinoma cell line (RPMI 2650) is used. The culture medium is minimum essential medium (MEM), supplemented with fetal bovine serum (FBS), sodium pyruvate, and non-essential amino acids (NEAA).

A cell suspension is prepared at a density of 2.5×10⁶ cells/mL, and 0.25 mL of the cell suspension is added to the insert of a Transwell plate. The cells are incubated at 37° C. with 5% CO₂ for 24 hours to allow adherence to the bottom of the insert. The medium in the insert is then removed to enable air-liquid interface (ALI) culture for 14 days, allowing the cells to differentiate into a monolayer. Fresh medium is replaced every 2-3 days, and transepithelial electrical resistance (TEER) is measured daily. After 14 days of culture, the formation of the nasal epithelial cell layer model is confirmed using a Millicell® ERS-2 volt-ohm meter. A TEER value of 120Ω is considered indicative of successful model establishment.

Regarding the corresponding drug doses, as described above, the drug concentration for the free diazoxide group is calculated based on the drug loading capacity (L.C.) of the diazoxide-loaded micelles to serve as a reference for comparison. As shown in Table 3, which indicates the drug concentration equivalents corresponding to the diazoxide-loaded micelles.

TABLE 3

| | PPP-D | MEM | Diazoxide |
|---|---|---|---|
| No. | (mL) | (mL) | (μg/mL) |
| 1 | 0.654 | 0.346 | 20 |
| 2 | 0.327 | 0.673 | 10 |
| 3 | 0.163 | 0.837 | 5 |
| 4 | 0.082 | 0.918 | 2.5 |
| 5 | 0.041 | 0.959 | 1.25 |
| 6 | 0.020 | 0.980 | 0.625 |

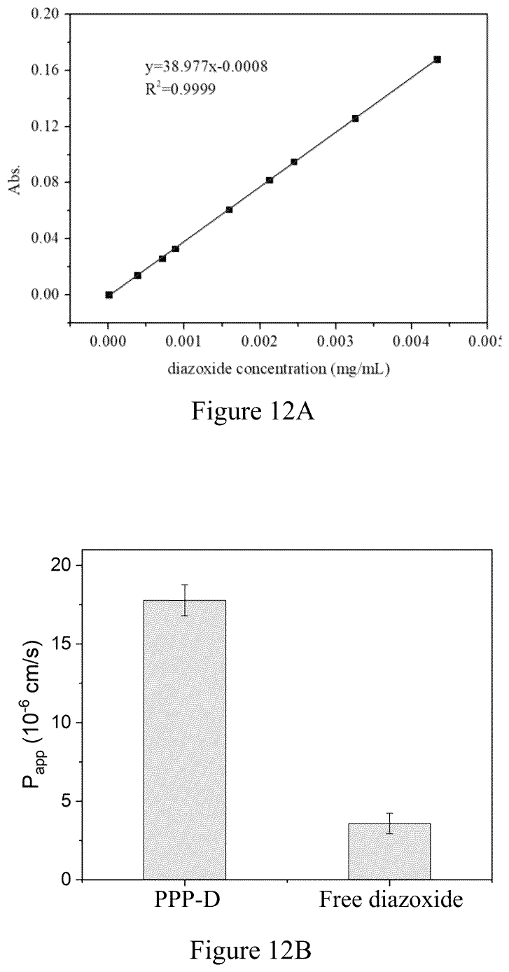

Next, corresponding to No. 1 in Table 3, after replacing the old culture medium and washing, diazoxide-loaded micelles are added to the inserts as a first experimental group, while free diazoxide is added to the inserts as a second experimental group. The cells are incubated at 37° C. with 5% CO₂ for 4 hours. In both the first and second experimental groups, 1 mL of phosphate-buffered saline (PBS) is added to the wells to collect components permeating through the nasal epithelial cell layer. After 4 hours of incubation, the PBS from the wells is collected, and its absorbance is measured using a UV-visible spectrophotometer. The absorbance values are then correlated with the drug calibration curve in PBS (as shown in FIG. 12A) to determine the drug concentration. The apparent permeability (Pₐₚₚ) is subsequently calculated using Equation 3.

$${P}_{a p p} = \frac{{Δ [ C ]}_{A} \times {V}_{A}}{A \times {[ C ]}_{D} \times Δ t} \qquad Equation 3$$

In equation 3, Δ[C]_{A} (mg/mL) represents the concentration difference in the well solution, [C]_{D} (mg/mL) represents the initial concentration in the well, Vₐ (mL) represents the total volume of the solution in the well (i.e., 1 mL), A (cm²) is the surface area of the nasal epithelial cell layer (1.13 cm²), and At is the permeation time (seconds, sec).

Referring to FIG. 12B, the apparent permeability (Pₐₚₚ, 10⁻⁶ cm/s) of the diazoxide-loaded micelles is approximately 4-fold higher than that of the free diazoxide.

The result shows that diazoxide encapsulated in the micelle passes more easily through the nasal epithelial cell layer, thereby facilitating its entry into the nose-to-brain pathway.

### Experiment 3

The composition comprising a potassium channel opener and a micelle can cross the blood-brain barrier via the nose-to-brain pathway, thereby achieving therapeutic effects on L-DOPA-induced dyskinesia and modulating endocrine functions.

The diazoxide-loaded micelles used in this experiment are prepared as described in Experiment 2. The animals used are 8-week-old male C57BL/6 mice, maintained under controlled temperature, humidity, and light-dark cycle conditions. Unless otherwise specified, food and water are provided ad libitum.

In some embodiments of this experiment, assessment of abnormal involuntary movements (AIMs) in mice involves both qualitative and quantitative evaluation of motor behaviors, including impairments of forelimb and axial movements. During testing, following administration of L-DOPA, mice are placed in transparent observation cylinders for at least 140 minutes, and their movements are recorded every 20 minutes. The detailed scoring method for abnormal involuntary movements is as described in Experiment 1. During the treatment period, scores for various types of dyskinesia are summed to yield a total abnormal involuntary movement score (total AIM score), which is used for subsequent data analysis.

In some embodiments of this experiment, techniques such as Western blotting, immunohistochemistry (IHC) staining, immunofluorescence (IF) staining, and gene expression analysis including RT-qPCR are employed. Those skilled in the art are familiar with these techniques, and thus detailed protocols are omitted herein.

### First Embodiment: Verification of Nose-to-Brain Delivery of Diazoxide-Loaded Micelles

To verify the delivery pathway of diazoxide-loaded micelles, a microliter pipette is used to administer the micelle solution into the mouse nostrils, allowing natural inhalation of the solution into the nasal cavity. The administered volume per nostril is 12.5 μL (total 25 μL per mouse; if 125 μL per nostril is intended, keep your original number), corresponding to a total dose of 0.04 mg/kg of diazoxide-loaded micelles.

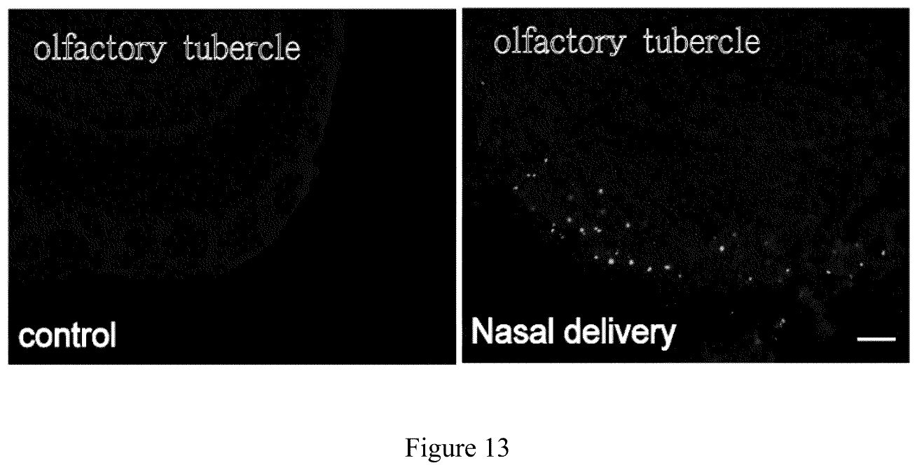

Subsequently, the olfactory tubercle region of the mice is isolated and subjected to immunofluorescence staining to evaluate whether the diazoxide-loaded micelles have migrated to this region. Referring to FIG. 13, the left panel shows the control group, which does not receive diazoxide-loaded micelles and accordingly shows no fluorescent signal. The right panel shows the experimental group, which receives diazoxide-loaded micelles and exhibits fluorescence. It should be understood that the olfactory tubercle is located in the forebrain and is part of the central nervous system.

### Second Embodiment: Therapeutic Effects of Diazoxide-Loaded Micelles in an L-DOPA-Induced Dyskinesia Model

To verify the therapeutic effects of diazoxide-loaded micelles, a unilateral nigrostriatal lesion mouse model is first established to induce Parkinson's disease-like pathology. Based on this model, an L-DOPA-induced dyskinesia (LID) mouse model is then established.

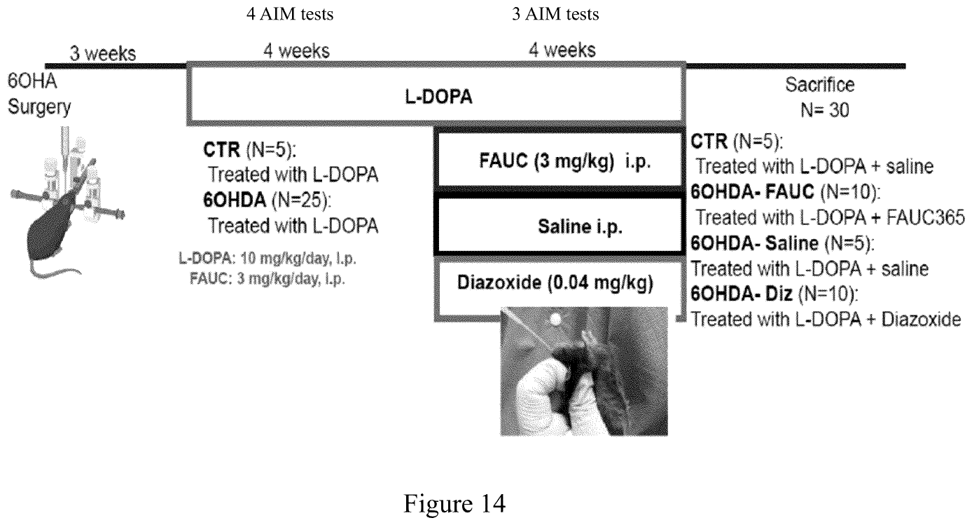

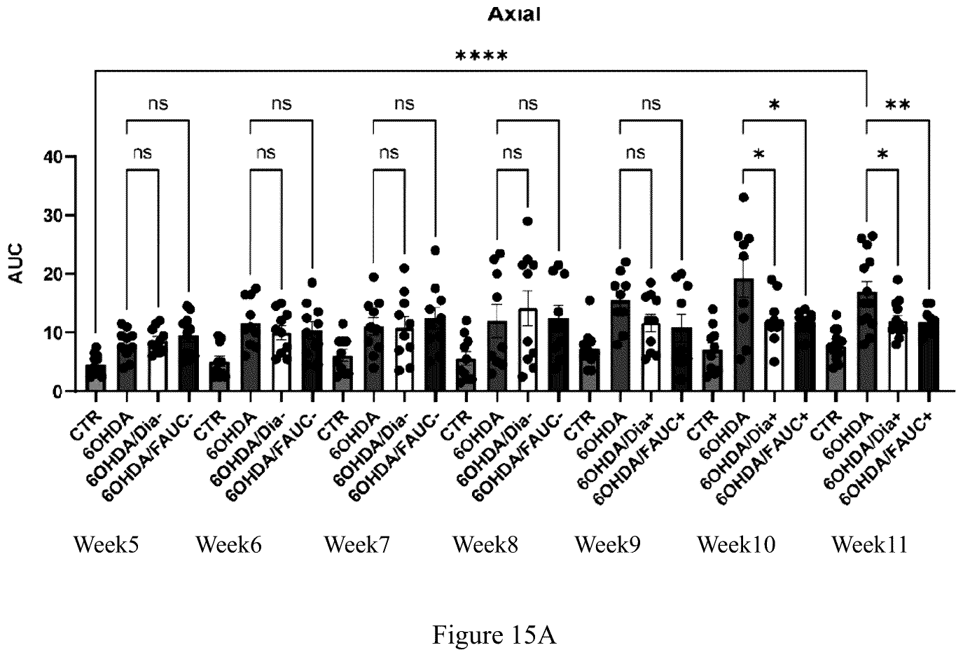

Referring to FIG. 14, mice are anesthetized with isoflurane prior to induction of the nigrostriatal lesion and then receive 6-hydroxydopamine (6-OHDA). Before administration, 6-OHDA is dissolved in saline containing 0.02% ascorbic acid at a concentration of 4 μg/μL. The injection is performed using a 10 μL syringe at coordinates relative to bregma (in mm): anteroposterior +0.14, lateral +2.1, and dorsoventral −3.5, at a rate of 1 μL/min. For establishment of the LID mouse model, L-DOPA is administered at 10 mg/kg per dose, once daily, for 4 weeks to induce involuntary movements.

In the experimental group, diazoxide-loaded micelles are administered to LID mice at a dose of 0.04 mg/kg via nasal administration, once daily for 4 weeks. In the control treatment group, FAUC-365, a known dopamine D3 receptor antagonist with efficacy against dyskinesia, is administered to another set of LID mice at 3 mg/kg via intraperitoneal injection, once daily for 4 weeks. Both the experimental and FAUC-365 groups receive L-DOPA as described above.

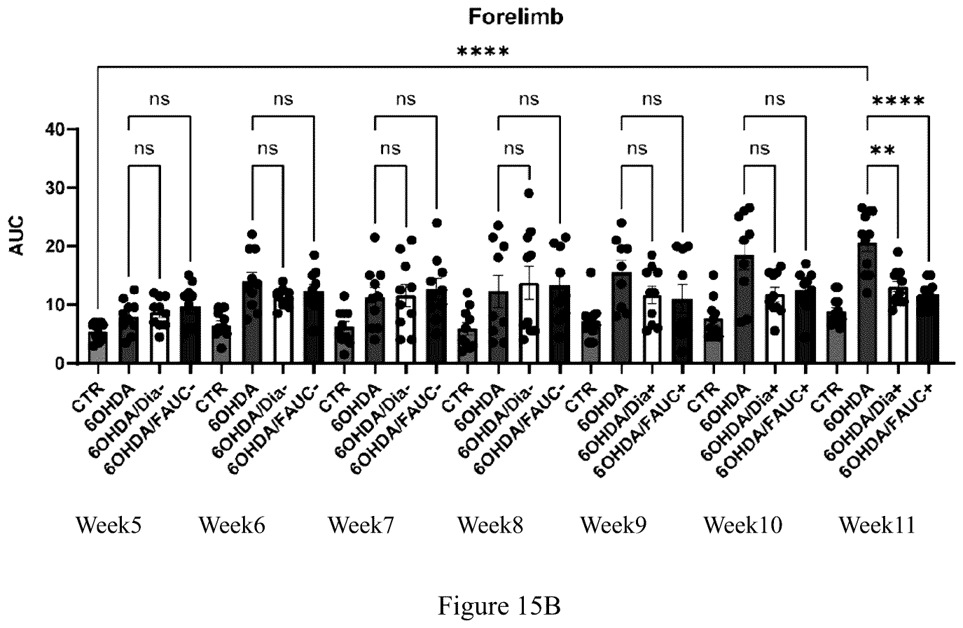

Referring to FIGS. 15A and 15B, axial and forelimb AIM scores are measured. It can be appreciated that at later time points, both diazoxide-loaded micelles administered via the nasal route and FAUC-365 administered via intraperitoneal injection effectively alleviate symptoms of involuntary movements.

To further demonstrate the neuroprotective effects of diazoxide-loaded micelles in the 6-OHDA-induced lesion model, immunohistochemistry is used to observe the distribution of tyrosine hydroxylase (TH) in the striatum (STR) and substantia nigra (SN) of mice. It should be understood that TH is the rate-limiting enzyme in dopaminergic neurons responsible for dopamine production. In the lesion group, the left hemisphere of the brain is injected with 6-OHDA at a concentration of 4 μg/μL, serving as the lesioned side, whereas the right hemisphere is injected with saline containing 0.02% ascorbic acid, serving as the non-lesioned side. In the treatment group, diazoxide-loaded micelles are administered to the mice of the lesion group. In the control (sham) group, both hemispheres are injected only with saline containing 0.02% ascorbic acid, without inducing any lesion.

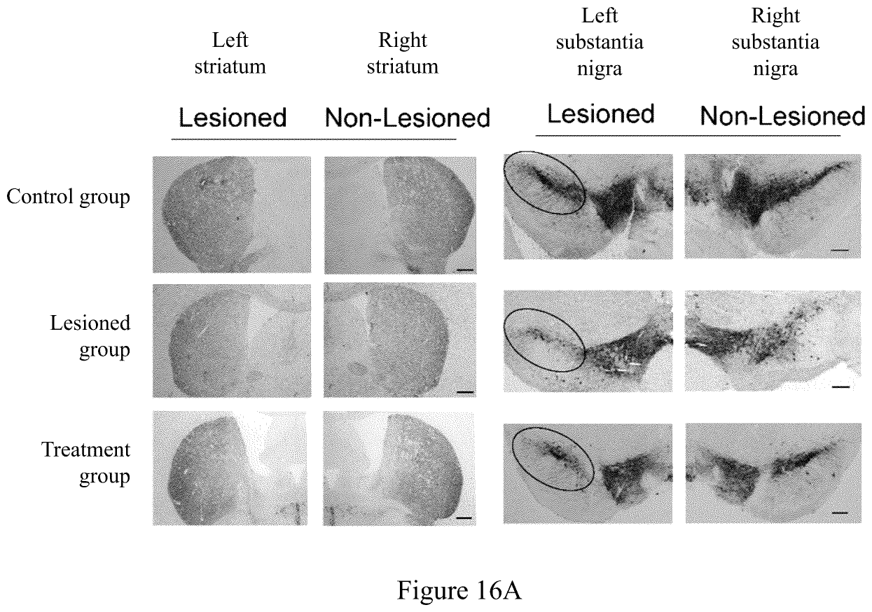

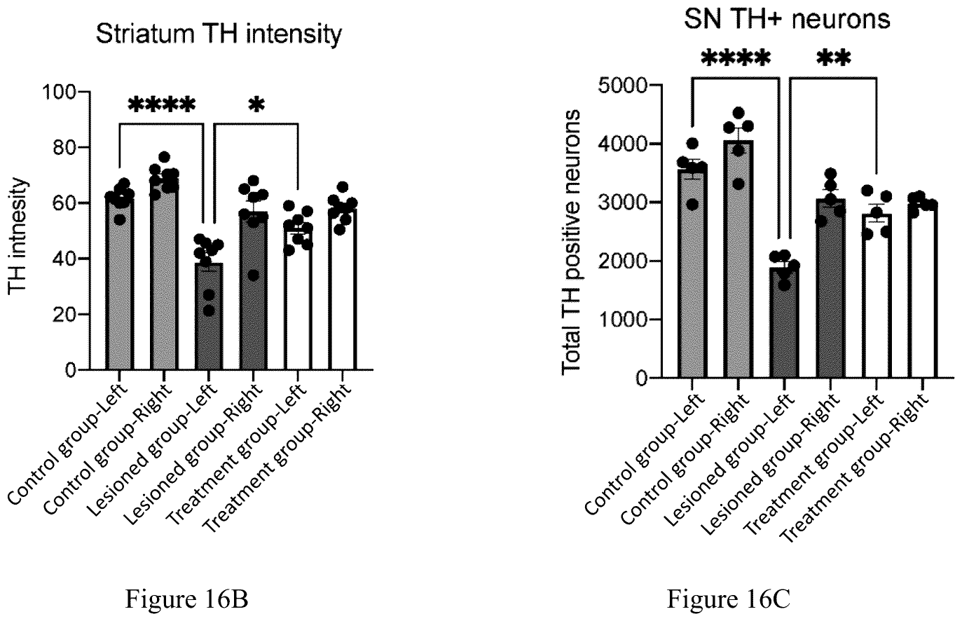

Referring to the brain images in FIG. 16A and the quantification in FIG. 16B, TH expression in both the left and right striatum of the control group is normal, indicating a higher number of synapses and neurons. By contrast, in the lesioned side of the lesion group, TH levels are significantly reduced compared with the control group. Further observation of the treatment group reveals that nasal administration of diazoxide-loaded micelles increases TH levels in the lesioned side, approaching those of the control group. This indicates recovery of synapses and neuronal numbers, as well as reversal of lesion-induced damage.

Therefore, these results suggest that diazoxide-loaded micelles can prevent neuronal loss and/or further degeneration.

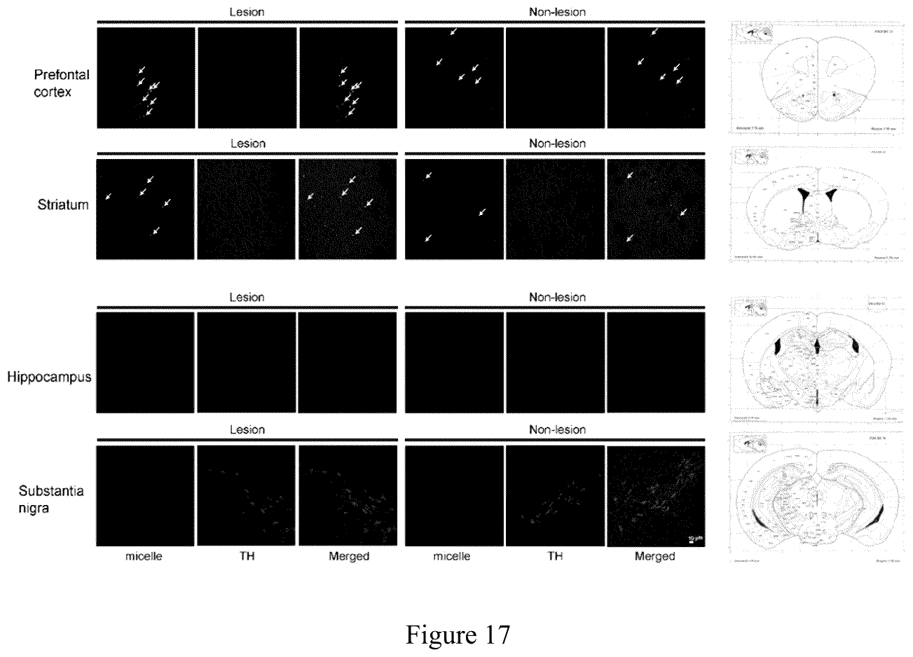

To further understand the brain distribution of diazoxide-loaded micelles, at the endpoint of the animal experiment, mouse brains are harvested for TH immunofluorescence staining. Serial coronal sections from the frontal cortex to the cerebellum are collected. Referring to FIG. 17, green fluorescent signals representing diazoxide-loaded micelles are mainly detected in the frontal brain regions, with stronger signals concentrated in the prefrontal cortex of the lesioned hemisphere, indicating higher uptake of the diazoxide-loaded micelles. Along the nigrostriatal pathway, green fluorescence is observed in the striatum, corresponding to TH distribution, indicating that diazoxide-loaded micelles reach the dopaminergic terminals. However, green fluorescence is not detected in dopaminergic neurons of the substantia nigra.

Accordingly, diazoxide-loaded micelles administered via the nasal route primarily accumulate in the frontal cortex and striatum. These results suggest their potential role in modulating dopaminergic function and in alleviating L-DOPA-induced dyskinesia.

### Third Embodiment: Effects of Diazoxide-Loaded Micelles on Glucose Homeostasis

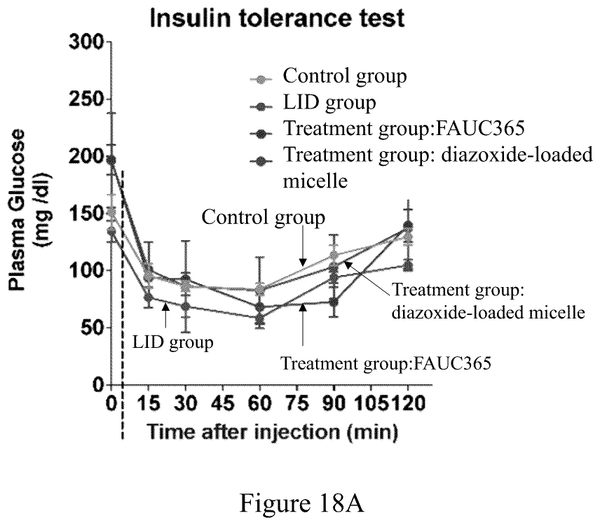

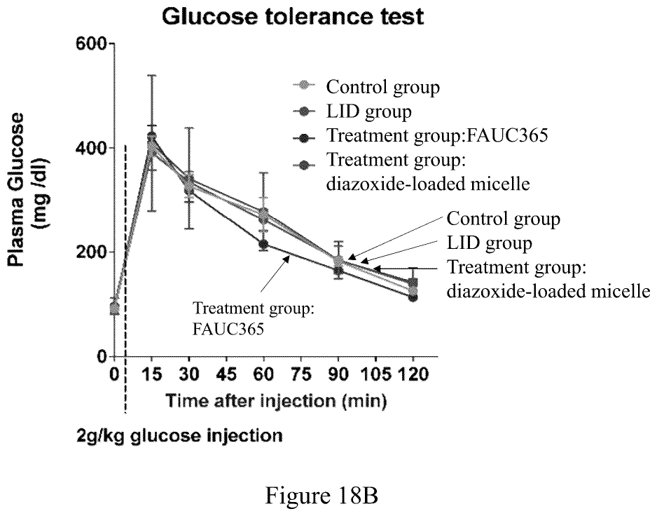

To evaluate the effects of diazoxide-loaded micelles on glucose homeostasis, insulin tolerance tests (ITT) and glucose tolerance tests (GTT) are performed. Referring to FIG. 18A, after administration of insulin (0.75 unit/kg) for 30-60 minutes, plasma glucose levels decrease in all groups. Notably, plasma glucose levels in the LID group decrease to a greater extent, indicating increased insulin sensitivity in these mice.

Referring to FIG. 18B, glucose tolerance test results show that plasma glucose levels in all groups peak at 15 minutes and then gradually decline. The glucose clearance profiles are similar across groups, indicating that administration of FAUC-365 or nasal delivery of diazoxide-loaded micelles normalizes insulin sensitivity in LID mice.

These results demonstrate that although LID mice exhibit higher insulin sensitivity, administration of FAUC-365 or nasal delivery of diazoxide-loaded micelles restores insulin sensitivity to normal levels, and no significant effect is observed on glucose tolerance (no statistical differences between groups). In other words, metabolic regulation in LID mice is maintained within normal limits following treatment with FAUC-365 or diazoxide-loaded micelles.

The aforementioned embodiments are merely exemplary of the present invention. It will be appreciated by those skilled in the art that various modifications, equivalents, and alterations may be made without departing from the technical concept of the present invention. Such modifications should likewise be regarded as falling within the scope of the present invention.

What is claimed is:

1. A composition for preventing or treating an involuntary movement related to dopaminergic dysfunction, comprising: a potassium channel opener and a pharmaceutically acceptable carrier, wherein the pharmaceutically acceptable carrier is a micelle.

2. The composition as claimed in claim 1, wherein the involuntary movement related to dopaminergic dysfunction is L-DOPA-induced dyskinesia.

3. The composition as claimed in claim 1, wherein the composition is formulated for administration through a nasal cavity.

4. The composition as claimed in claim 3, wherein the composition is in the form of a liquid, a dry powder, a suspension, or any combination thereof.

5. The composition as claimed in claim 1, wherein the potassium channel opener comprises an ATP-sensitive potassium (K_{ATP}) channel opener.

6. The composition as claimed in claim 5, wherein the ATP-sensitive potassium (K_{ATP}) channel opener comprises diazoxide.

7. The composition as claimed in claim 1, wherein the potassium channel opener is embedded in or encapsulated by the micelle.

8. The composition as claimed in claim 1, wherein the micelle comprises a block copolymer.

9. The composition as claimed in claim 8, wherein:
the particle size of the micelle is between 35 nm and 70 nm; and/or
the zeta potential of the micelle is between −15 mV and −2 mV.

10. The composition as claimed in claim 1, wherein the weight ratio of the potassium channel opener to the micelle ranges from 1:100 to 1:1500.

11. A method for preventing or treating an involuntary movement related to dopaminergic dysfunction, comprising: administering to a subject in need a composition comprising a potassium channel opener and a pharmaceutically acceptable carrier, wherein the pharmaceutically acceptable carrier is a micelle.

12. The method as claimed in claim 11, wherein the involuntary movement related to dopaminergic dysfunction is L-DOPA-induced dyskinesia.

13. The method as claimed in claim 11, further comprising: administering the composition through a nasal cavity of the subject in need.

14. The method as claimed in claim 13, wherein the composition is in the form of a liquid, a dry powder, a suspension, or any combination thereof.

15. The method as claimed in claim 11, wherein the potassium channel opener comprises an ATP-sensitive potassium (K_{ATP}) channel opener.

16. The method as claimed in claim 15, wherein the ATP-sensitive potassium (K_{ATP}) channel opener comprises diazoxide.

17. The method as claimed in claim 11, wherein the potassium channel opener is embedded in or encapsulated by the micelle.

18. The method as claimed in claim 11, wherein:
the micelle comprises a block copolymer;
the particle size of the micelle is between 35 nm and 70 nm; and/or
the zeta potential of the micelle is between −15 mV and −2 mV.

19. The method as claimed in claim 11, wherein the weight ratio of the potassium channel opener to the micelle ranges from 1:100 to 1:1500.

20. The method as claimed in claim 11, further comprising:
administering the composition at a dose from 0.08 μg/kg to 120 μg/kg to the subject in need, wherein the subject in need is a human.
